(19) United States
(12) Patent Application Publication
(10) Pub. No.: US 2015/0334276 A1
(43) Pub. Date: Nov. 19, 2015

(54) SYSTEM AND METHOD FOR DISPLAYING AN IMAGE STREAM

(71) Applicant: GIVEN IMAGING LTD., Yoqneam (IL)

(72) Inventors: Ady ECKER, Nes-Ziona (IL); Hagai KRUPNIK, Nofit (IL)

(21) Appl. No.: 14/758,400

(22) PCT Filed: Dec. 30, 2013
(86) PCT No.: PCT/IL2013/051081

§ 371 Date: Jun. 29, 2015

Related U.S. Application Data
(60) Provisional application No. 61/747,514, filed on Dec. 31, 2012.

(51) Int. Cl. H04N 5/225; G06T 5/50; A61B 1/00; H04N 5/341; G06T 3/00; A61B 1/04; G06T 3/40; H04N 7/18

(52) U.S. Cl. CPC H04N 5/2252; G06T 3/4038; G06T 5/50; H04N 5/2256; H04N 7/185; H04N 5/341; G06T 3/0093; A61B 1/041; A61B 1/0005; G06T 2200/32; G06T 2207/10004; G06T 2207/10016; G06T 2207/10024; G06T 2207/10068; G06T 2207/20164; G06T 2207/20221; G06T 2207/30092; G06T 2207/30196; G06T 2210/41; H04N 2005/2255

(57) ABSTRACT

A system and method to display an image stream captured by an in vivo imaging capsule may include displaying an image stream of consolidated images, the consolidated images generated from a plurality of original images. To generate the consolidated image, a plurality of original images may be mapped to a selected template, the template comprising at least a mapped image portion and a generated image portion. The generated image portion may be filled by copying a patch from the mapped image portion, and edges between the generated portion and the mapped image portion may be smoothed or blended. The smoothing is per formed by calculating offset values of pixels in the generated portion, and for each pixel in the generated portion, adding the calculated offset value of the pixel to the color value of the pixel.

### FIELD OF THE INVENTION

The present invention relates to a method and system for displaying and/or reviewing image streams. More specifically, the present invention relates to a method and system for effective display of multiple images of an image stream, generated for example by a capsule endoscope.

### BACKGROUND OF THE INVENTION

An image stream may be assembled from a series of still images and displayed to a user. The images may be created or collected from various sources, for example using Given Imaging Ltd.'s commercial PillCam® SB2 or ESO2 swallowable capsule products. For example, U.S. Pat. Nos. 5,604,531 and/or 7,009,634 to Iddan et al., assigned to the common assignee of the present application and incorporated herein by reference, teach an in-vivo imager system which in one embodiment includes a swallowable or otherwise ingestible capsule. The imager system captures images of a lumen such as the gastrointestinal (GI) tract and transmits them to an external recording device while the capsule passes through the lumen. The capsule may advance along lumen portions at different progress rates, moving at an inconsistent speed, which may be faster or slower depending on the peristaltic movement of the intestines. Large numbers of images may be collected for viewing and, for example, combined in sequence. Images may be selected for display from the original image stream, and a subset of the original image stream may be displayed to a user. The time it takes to review the complete set of captured images may be relatively long, for example may take several hours.

A reviewing physician may want to view a reduced set of images, which includes images which are important or clinically interesting, and which does not omit any relevant clinical information. The reduced or shortened movie may include images of clinical importance, such as images of selected predetermined locations in the gastrointestinal tract, and images with pathologies or abnormalities. For example, U.S. patent application Ser. No. 10/949,220 to Davidson et al., assigned to the common assignee of the present application and incorporated herein by reference, teaches in one embodiment a method of editing an image stream, for example by selecting images which follow predetermined criteria.

In order to shorten the review time, an original image stream may be divided into two or more subset images streams, the subset image streams being displayed simultaneously or substantially simultaneously. U.S. Pat. No. 7,505,062 to Davidson et al., assigned to the common assignee of the present application and incorporated herein by reference, teaches a method for displaying images from the original image stream across a plurality of consecutive time slots, wherein in each time slot a set of consecutive images from the original image stream is displayed, thereby increasing the rate at which the original image stream can be reviewed without reducing image display time. Post processing may be used to fuse images shown simultaneously or substantially simultaneously. Examples of fusing images can be found, for example, in embodiments described in U.S. Pat. No. 7,474,327, assigned to the common assignee of the present invention and incorporated herein by reference.

Displaying a plurality of subset image streams simultaneously may create a movie which is more challenging for a user to review, compared to reviewing a single image stream. For example, when viewing a plurality of subset image streams simultaneously, the images are typically displayed at a faster total rate, and the user needs to be more focused, concentrated, and alert to possible pathologies being present in the multiple images displayed simultaneously.

### SUMMARY OF THE INVENTION

A system and method to display an image stream captured by an in vivo imaging capsule may include generating a consolidated image, the consolidated image comprising a mapped image portion and a generated portion. The mapped image portion may comprise boundary pixels, which indicate the boundary between the mapped portion and the generated portion of the consolidated image. The generated portion may comprise pixels adjacent to the boundary pixels and internal pixels.

A distance transform for the pixels of the generated portion may be performed, and for each pixel, the distance of the pixel to the nearest boundary pixel may be calculated. Offset values of pixels in the generated portion may be calculated. Offset values of a pixel P_{A} in the generated portion, adjacent to a boundary pixel, may be calculated, for example, by computing the difference between a color value of P_{A} and a mean, median, generalized mean or weighted average of at least one neighboring pixel. The neighboring pixel may be selected from the boundary pixels adjacent to P_{A}.

In some embodiments, offset values of internal pixels in the generated portion may be calculated based on the offset values of at least one neighboring pixel which had been assigned an offset value. For example, calculating offset values of an internal pixel in the generated portion may be performed by computing a mean, median, generalized mean or weighted average of at least one neighboring pixel which has been assigned an offset value, times a decay factor.

For each pixel in the generated portion, the calculated offset value of the pixel may be added to the color value of the pixel, to obtain a new pixel color value. The consolidated image comprising the mapped image portion and the generated portion with the new pixel color values may be displayed.

The method may include receiving a set of original images from an in vivo imaging capsule for concurrent display, and selecting a template for displaying the set of images. The template may comprise at least a mapped image portion and a generated portion. The original images may be mapped to the mapped image portion in the selected template. A fill may be generated or synthesized, for predetermined areas of the consolidated image (e.g. according to a selected template), to produce the generated portion of the consolidated image. Generating the fill may be performed by copying a patch from the mapped image portion to the generated portion.

Pixels in the generated portion may be sorted, for example based on the calculated distance, and the offset values of internal pixels may be calculated according to the sorted order. The boundary pixels of the mapped image portion may comprise pixels which are adjacent pixels of the corresponding generated portion.

Embodiments of the present invention may include a system for displaying a consolidated image, the consolidated image may comprise at least a mapped image portion and a generated portion. The mapped image portion may comprise boundary pixels, and the generated portion may comprise pixels adjacent to the boundary pixels and internal pixels. The system may include a processor to calculate, e.g. for pixels of the generated portion, a distance value of the pixel to the nearest boundary pixel. The processor may calculate offset values of the pixels of the generated portion which are adjacent the boundary pixels. Offset values of internal pixels in the generated portion may be calculated based on the offset values of at least one neighboring pixel which had been assigned an offset value. For each pixel in the generated portion, the calculated offset value of the pixel may be added to the color value of the pixel to obtain a new pixel color value. The system may include a storage unit to store the distance values, the offset values, and the new pixel color values, and a display to display the consolidated image, the consolidated image comprising the mapped image portion and the generated portion with the new pixel color values.

In some embodiments, the storage unit may store a set of original images from an in vivo imaging capsule for concurrent display. The processor may to select a template for displaying the set of images. The template may comprise at least a mapped image portion and a generated portion. The processor may to map the original images to the mapped image portion in the selected template to produce the mapped image portion. The processor may generate fill for predetermined areas of the consolidated image to produce the generated portion. For example, the fill may be generated by copying a patch from the mapped image portion to the generated portion.

In some embodiments, the processor may sort pixels in the generated portion based on the calculated distance value, and to calculate the offset values of internal pixels according to the sorted order.

Embodiments of the invention include a method of deforming multiple images of a video stream to fit a human field of view. Distortion minimization technique may be used to deform an image to a new contour based on a template pattern, the template pattern having rounded corners and an oval-like shape. The deformed images may be displayed as a video stream. The template pattern may include a mapped image portion and a synthesized portion. The values of the synthesized portion may be calculated by copying a region of the mapped image portion to the synthesized portion, and smoothing the edges between the mapped image portion and the synthesized portion.

### BRIEF DESCRIPTION OF THE DRAWINGS

The present invention will be understood and appreciated more fully from the following detailed description taken in conjunction with the drawings in which:

FIG. 1 shows a schematic diagram of an in-vivo imaging system according to an embodiment of the present invention;

FIG. 2 depicts an exemplary graphic user interface display of an in vivo image stream according to an embodiment of the present invention;

FIGS. 3A-3C depict exemplary dual image displays according to embodiments of the invention;

FIG. 3D depicts an exemplary dual image template according to an embodiment of the present invention

FIG. 4 depicts an exemplary triple image display according to embodiments of the invention;

FIG. 5 depicts an exemplary quadruple image display according to embodiments of the invention;

FIG. 6 is a flowchart depicting a method for displaying a consolidated image according to an embodiment of the invention;

FIG. 7A is a flowchart depicting a method for generating a predetermined empty portion in a consolidated image according to an embodiment of the invention;

FIG. 7B is a flowchart depicting a method for smoothing edges of a generated portion in a consolidated image according to an embodiment of the invention; and

FIG. 7C is an enlarged view of the top left portion of the consolidated quadruple image display shown in FIG. 5.

### DETAILED DESCRIPTION OF THE INVENTION

In the following description, various aspects of the present invention will be described. For purposes of explanation, specific configurations and details are set forth in order to provide a thorough understanding of the present invention. However, it will also be apparent to one skilled in the art that the present invention may be practiced without the specific details presented herein. Furthermore, well-known features may be omitted or simplified in order not to obscure the present invention.

A system and method according to one embodiment of the invention enable a user to see images of an image stream for a longer period of time without increasing the overall viewing time of the edited image stream. Alternatively, the system and method described according to one embodiment may be used to increase the rate at which a user can review an image stream without sacrificing details that may be depicted in the stream. In certain embodiments, the images are collected from a swallowable or otherwise ingestible capsule traversing the GI tract. The images may be combined into an image stream or movie. In some embodiments, an original image stream or complete image stream may be created, that includes all images (e.g., complete set of frames) captured or received during the imaging procedure. A plurality of images from the image stream may be displayed simultaneously or substantially simultaneously on a screen or monitor.

In other embodiments a reduced or edited image stream may include a selection of the images (e.g., subset of the captured frames), selected according to one or more predetermined criteria. In some embodiments, images may be omitted from an original image stream, e.g. an original image stream may include less images than the number of images captured by the swallowable capsule. For example, images which are oversaturated, blurred, include intestinal contents or turbidity, and/or images which are very similar to neighboring images, may be removed from the full set of images captured by the imaging capsule, and an original image stream may include a subset of the images captured by the imaging capsule. In such cases, a reduced image stream may include a reduced subset of images selected from the original image stream according to predetermined criteria.

Embodiments of the invention may include an article such as a computer or processor readable non-transitory storage medium, such as for example a memory, a disk drive, or a USB flash memory device encoding, including or storing instructions, e.g., computer-executable instructions, which when executed by a processor or controller, cause the processor or controller to carry out methods disclosed herein.

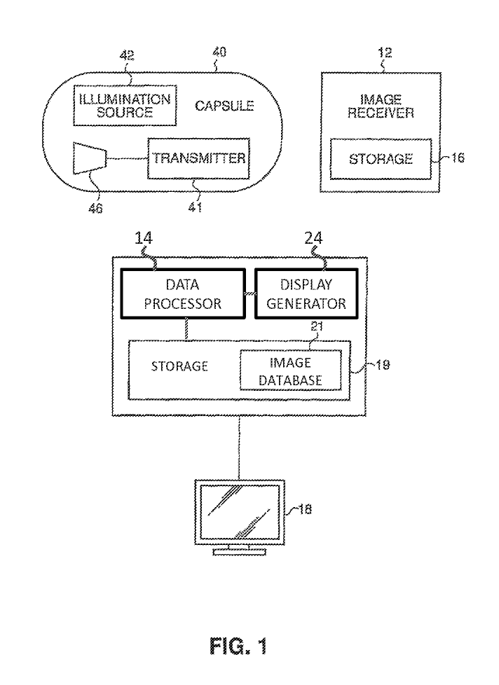

Reference is made to FIG. 1, which shows a schematic diagram of an in-vivo imaging system according to one embodiment of the present invention. In an exemplary embodiment, the system includes a capsule 40 having one or more imagers 46, for capturing images, one or more illumination sources 42, for illuminating the body lumen, and a transmitter 41, for transmitting image and possibly other information to a receiving device. The in vivo imaging device may correspond to embodiments described in U.S. Pat. No. 5,604,531 and/or in U.S. Pat. No. 7,009,634 to Iddan et al, and/or in U.S. patent application Ser. No. 11/603,123 to Gilad, but in alternate embodiments may be other sorts of in vivo imaging devices. The images captured by the imaging system may be of any suitable shape including for example circular, square, rectangular, octagonal, hexagonal, etc. Typically, located outside the patient's body in one or more locations are an image receiver 12, including an antenna or antenna array (not shown), an image receiver storage unit 16, a data processor 14, a data processor storage unit 19, and an image monitor 18, for displaying, inter alia, images recorded by the capsule 40. Typically, data processor storage unit 19 includes an image database 21. Processor 14 and/or other processors, or image display generator 24, may be configured to carry out methods as described herein by, for example, being connected to instructions or software stored in a storage unit or memory which when executed by the processor cause the processor to carry out such methods.

Typically, data processor 14, data processor storage unit 19 and monitor 18 are part of a personal computer or workstation, which includes standard components such as processor 14, a memory, a disk drive, and input-output devices such as a mouse and keyboard, although alternate configurations are possible. Data processor 14 may include any standard data processor, such as a microprocessor, multiprocessor, accelerator board, or any other serial or parallel high performance data processor. Data processor 14 typically, as part of its functionality, acts as a controller controlling the display of the images (e.g., which images, the location of the images among various windows, the timing or duration of display of images, etc.). Image monitor 18 is typically a conventional video display, but may, in addition, be any other device capable of providing image or other data. The image monitor 18 presents the image data, typically in the form of still and moving pictures, and in addition may present other information. In an exemplary embodiment, the various categories of information are displayed in windows. A window may be for example a section or area (possibly delineated or bordered) on a display or monitor; other windows may be used. Multiple monitors may be used to display image and other data, for example an image monitor may also be included in image receiver 12. Data processor 14 or other processors may carry out methods as described herein. For example, image display generator 24 or other modules may be software executed by data processor 14, or may be processor 14 or another processors, for example executing software or controlled by dedicated circuitry.

In operation, imager 46 captures images and sends data representing the images to transmitter 41, which transmits images to image receiver 12 using, for example, electromagnetic radio waves. Image receiver 12 transfers the image data to image receiver storage unit 16. After a certain period of time of data collection, the image data stored in storage unit 16 may be sent to the data processor 14 or the data processor storage unit 19. For example, the image receiver 12 or image receiver storage unit 16 may be taken off the patient's body and connected to the personal computer or workstation which includes the data processor 14 and data processor storage unit 19 via a standard data link, e.g., a serial, parallel, USB, or wireless interface of known construction. The image data is then transferred from the image receiver storage unit 16 to an image database 21 within data processor storage unit 19. Typically, the image stream is stored as a series of images in the image database 21, which may be implemented in a variety of known manners.

Data processor 14 may analyze the data and provide the analyzed data to the image monitor 18, where a user views the image data. Data processor 14 operates software that, in conjunction with basic operating software such as an operating system and device drivers, controls the operation of data processor 14. Typically, the software controlling data processor 14 includes code written in the C++ language, and may be implemented using various development platforms such as Microsoft's .NET platform, but may be implemented in a variety of known methods.

Data processor 14 may include or execute graphics software and/or hardware. Data processor 14 may assign one or more scores, ratings or measures to each frame based on a plurality of pre-defined criteria. When used herein, a “score” may be a general score or rating, where (in one embodiment) the higher the score the more likely a frame is to be included in a movie, and (in another embodiment) a score may be associated with a specific property, e.g., a quality score, a pathology score, a similarity score, or another score or measure that indicates an amount or likelihood of a quality a frame has. The data processor 14 may select the frames with scores within an optimal range for display and/or remove those with scores within a sub-optimal range. The scores may represent, for example, a (normal or weighted) average of the frame values or sub-scores associated with the plurality of pre-defined criteria. The subset of selected frames may be played, in sequence, as an edited (reduced) movie or image stream.

The images in an original stream and/or in a reduced stream may be sequentially ordered (and thus the streams may have an order) according to the chronological time of capture, or may be arranged according to different criteria (such as degree of similarity between images, color levels, illumination levels, estimated distance of the object in the image from the in vivo device, suspected pathological rating of the images, etc.).

Data processor 14 may include, or may be operationally connected to, an image display generator 24. The image display generator 24 may be used for generating a single consolidated image for display from a plurality of images. For example, image display generator 24 may receive a plurality of original image frames e.g., an image stream), e.g. from image database 21, and generate a consolidated image which comprises the plurality of image frames.

An original image frame, as used herein, refers to a single image frame which was captured by an imager, e.g. an in vivo imaging device. In some embodiments, the original image frames may undergo certain image pre-processing operations, such as centering, normalizing the intensity of the image, unifying the shape and size of the image, etc.

A consolidated image, as used herein, is a single image composed of a plurality of images such as original images captured by the capsule 40. Each image in the consolidated image may have been captured at a different time. The consolidated image typically has a predetermined shape or contour (e.g., defined by a template). The predetermined shape or contour of the template pattern is designed to better fit the human field of view, using a circular or oval-like shape. The template pattern is formed such that all the visual data which is captured in the original images is conveyed or displayed to the user, and no (substantial or noticeable) visual data is lost or removed. Since the human field of view is rounded, it may be difficult to view details which are positioned in the corners of a consolidated image, e.g. if the consolidated image was rectangular.

Each of the original images which compose the consolidated image may be mapped to a predetermined region in the consolidated image. The shape or contour of the original image is typically different from the shape or contour of the region in the consolidated image to which the original image is mapped.

A user may select the number of original images to be displayed as a single consolidated image. Based on the selected number of images (e.g. 1, 2, 3, 4, 16) which are to be displayed simultaneously, a single consolidated image may be generated. Image display generator 24 may map the selected number of original images to the predetermined regions in a consolidated image, and may generate consolidated images for display as an image stream.

In some embodiments, image display generator 24 may determine properties of the displayed consolidated image, e.g. the position and size on screen, the shape and/or contour of a consolidated image generated from a plurality of original images, the automatic generation and application to an image of image content to fill certain predetermined areas of the template, and/or generating the border between the mapped images. If the user selected, for example, four images to be displayed simultaneously, image display generator 24 may determine, create or choose the template (which may include the contour or outline shape and size of the consolidated image (e.g. from a list of stored templates), select four original images from the stream, and map the four original images according to four predetermined regions of the consolidated image template to generate a single consolidated image. This process may be performed for the complete image stream, e.g. for all images in the originally captured image stream, or for portions thereof (e.g. for an edited image stream).

The image data (e.g., original image stream) collected and stored may be stored indefinitely, transferred to other locations, manipulated or analyzed. A health professional may, for example, use the images to diagnose pathological conditions or abnormalities of the GI tract, and, in addition, the system may provide information about the location of these pathologies. While, using a system where the data processor storage unit 19 first collects data and then transfers data to the data processor 14, the image data is not viewed in real time, other configurations allow for real time viewing, for example viewing the images on a display or monitor which is part of the image receiver 12.

The image data recorded and transmitted by the capsule 40 may be digital color image data, although in alternate embodiments other image formats may be used. In an exemplary embodiment, each frame of image data includes 320 rows of 320 pixels each, each pixel including bytes for color and brightness, according to known methods. For example, in each pixel, color may be represented by a mosaic of four sub-pixels, each sub-pixel corresponding to primaries such as red, green, or blue (where one primary may be represented twice). The brightness of the overall pixel may be recorded by a one byte (i.e., 0-255) brightness value. Images may be stored, for example sequentially, in data processor storage unit 19. The stored data is comprised of one or more pixel properties, including color and brightness. Other image formats may be used.

Data processor storage unit 19 may store a series of images recorded by a capsule 40. The images the capsule 40 records, for example, as it moves through a patient's GI tract may be combined consecutively to form a series of images displayable as an image stream. When viewing the image stream, the user is typically presented with one or more windows on monitor 18; in alternate embodiments multiple windows need not be used and only the image stream may be displayed. In an embodiment where multiple windows are provided, for example, an image window may provide the image stream, or still portions of that image. Another window may include buttons or other controls that may alter the display of the image; for example, stop, play, pause, capture image, step, fast-forward, rewind, or other controls. Such controls may be activated by, for example, a pointing device such as a mouse or trackball. Typically, the image stream may be frozen to view one frame, speeded up, or reversed; sections may be skipped; or any other method for viewing an image may be applied to the image stream.

In one embodiment, an original image stream, for example an image stream captured by an in vivo imaging capsule, may be edited or reduced according to different selection criteria. Examples of selection criteria disclosed, for example, in paragraph [0032] of U.S. Patent Application Publication Number 2006/0074275 to Davidson et al., assigned to the common assignee of the present application and incorporated herein by reference, include numerically based criteria, quality based criteria, annotation based criteria, color differentiation criteria and/or resemblance to a preexisting image such as an image depicting an abnormality. The edited or reduced image stream may include a reduced number of images compared to the original image stream. In some embodiments, a reviewer may view the reduced stream in order to save time, for example instead of viewing the original image stream.

When viewing an in vivo image stream, the display rate of the images may vary, for example according to the estimated speed of the in vivo device while capturing the images, or according to the similarity between consecutive images in the stream. For example, in an embodiment disclosed in U.S. Pat. No. 6,709,387, an image processor correlates at least two image frames to determine the extent of their similarity, and to generate a frame display rate correlated with said similarity, wherein said frame display rate is slower when said frames are generally different and faster when said frames are generally similar.

The image stream may be presented to the viewer by displaying a consolidated image in a single window, such that a set of consecutive or adjacent (e.g., next to each other in time, or in time of capture) frames in a complete image stream or in an edited image stream may be displayed substantially simultaneously. According to one embodiment, in each time slot (e.g. a period in which one or more images is to be displayed in a window), a plurality of images which are consecutive in the image stream are displayed as a single consolidated image. The duration of the timeslots may be uniform for all timeslots, or varying.

In an exemplary embodiment, in order to improve the visibility of pathologies and create a more suitable or comfortable view for the human field of view, image display generator 24 may map or warp the original images (to a predetermined shaped field) to create a smoother contour of the consolidated image. Such mapping may be performed, for example, using conformal mapping techniques (a transformation that preserves local angles, also called conformal transformation, angle-preserving transformation, or biholomorphic map) as known in the art. The template design of the mapped image portions may typically be symmetrical, e.g. each image may be displayed in similar or equal shape and size as the other original images which compose the consolidated image. For example, images may be reversed and presented as a mirror image, the images may have their orientation otherwise altered, or the images may be otherwise processed to increase symmetry. In one example, the original images may be circular, and the consolidated image may have a rounded-rectangular shape.

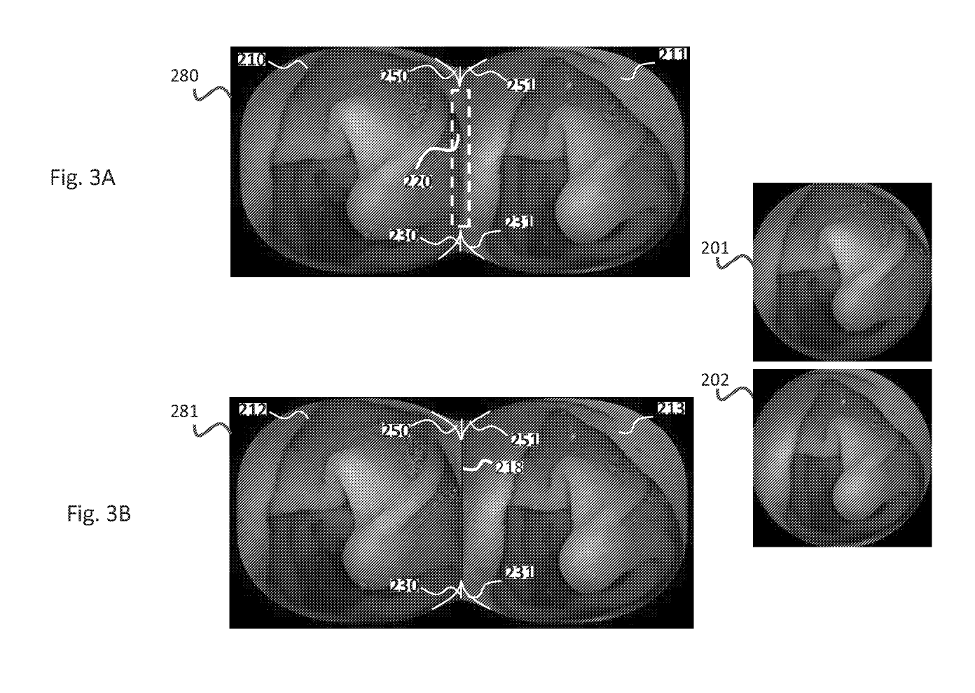

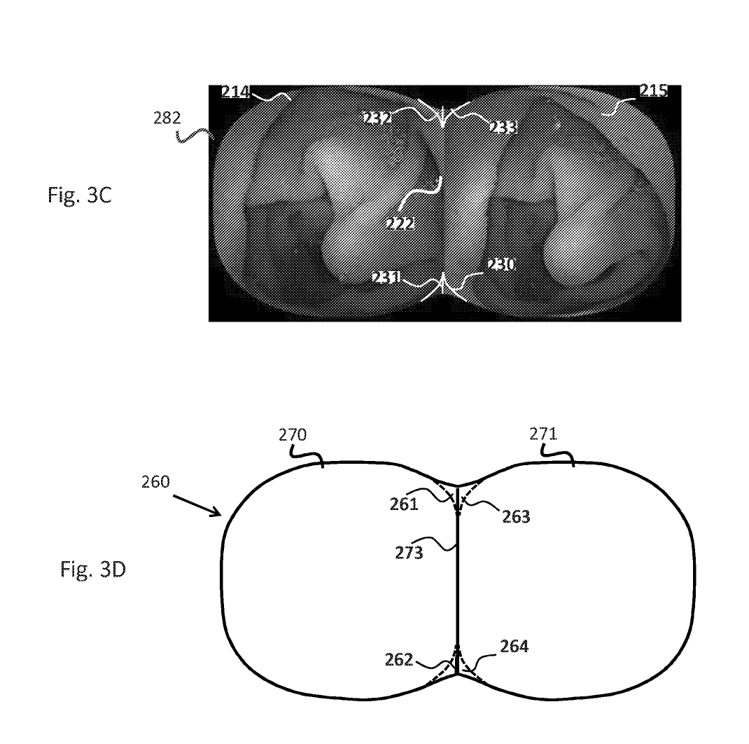

In some embodiments, the template for creating the consolidated image may include predetermined empty portions which are not filled by the distortion minimization technique (e.g. conformal mapping algorithm). In one example, the original image may be circular and the shape of the mapped region in the consolidated image may be square-like or similar to a rectangle with rounded corners. When applying the known distortion minimization techniques to the square-like region, the distortion minimization technique may generate large magnifications of image portions at the corners. Thus, embodiments of the present invention use a mapping template with corners which are rounded, and the empty portions (e.g. in the middle of the consolidated image and at the corners connecting the mapped images, as shown in FIG. 3D) which are not filled by the distortion minimization technique may be filled by other methods. In some embodiments, image display generator 24 may generate the fill for the predetermined empty portions of the consolidated image. A template may define how a set of images are to be placed and/or how the images are to be shaped or modified, when the images are displayed.

The viewing time of the image stream may be reduced when a plurality of images are displayed simultaneously. For example, if an image stream is generated from consolidated images, each consolidated image including two or more original images being displayed simultaneously, and in each consecutive time slot a consecutive consolidated image is displayed (e.g., with no repeated original images displayed in different time slots, such that each image is displayed in only one time slot), then the total viewing time of the image stream may be reduced to half of the original time, or the duration of each time slot may be longer to enable the reviewer more time to examine the images on display, or both may occur. For example, if an original image stream may be displayed at 20 frames per second, two images displayed simultaneously in each time slot may be displayed at 10 frames per second. Therefore the same number of overall frames per second is displayed, but the user can view twice as much information and each frame is displayed twice as long.

A trade-off exists between the total display time for the image stream and the duration that each image appears on display. For example, the total viewing time may be the same as that of the original image stream, but each frame is displayed to the user for a longer period of time. In another example, if a user is comfortably viewing a single displayed image at one rate, adding a second image will allow the user to increase the total review rate without reducing the time that each frame is displayed. In alternate embodiments, the relationship between the display rate when the image stream is displayed as a stream of single images and when it is displayed as a stream of consolidated image may differ; for example, the resulting consolidated image stream may be displayed at the same rate as the original image stream. Therefore, the display method may not only reduce a total viewing time of the image stream, but also increase the duration of display time of some or all images on the screen.

In an exemplary embodiment, the user may switch modes, between viewing a single image at each time slot and viewing multiple images at each time slot, for example using a control such as a keystroke or on-screen button selected using a pointing device (e.g., mouse or touchpad). The user may control the multiple image display in a manner similar to the control of a single image display, for example by using on screen controls.

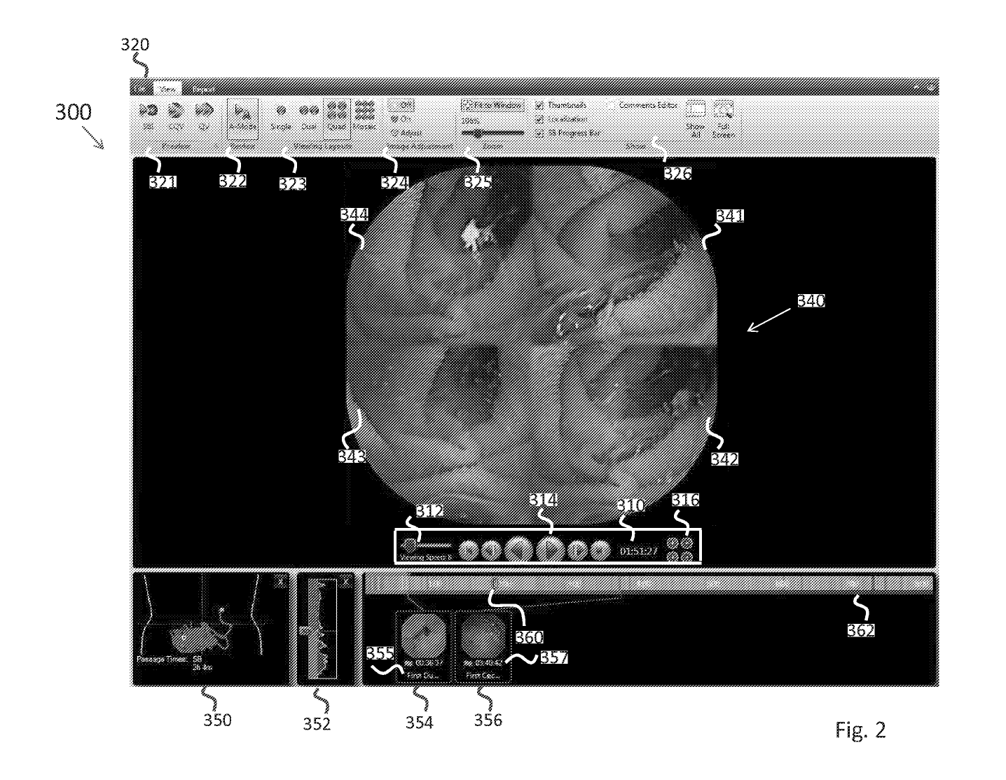

Reference is now made to FIG. 2, which depicts an exemplary graphic user interface display of an in vivo image stream according to an embodiment of the present invention. Display 300 includes various user interface options and an exemplary consolidated image stream window 340. The display 300 may be displayed on, for example, image monitor 18. Consolidated image stream window 340 may include a plurality of original images consolidated into a single window. The consolidated image may include a plurality of image portions (or regions) e.g. portions 341, 342, 343, 344. Each image portion or region may correspond to a different original image, e.g. a different image in the original captured image stream. The original images may be warped or mapped into the image portions 341-344, and may be fused together (e.g. with smoothed edges between the image portions 341-344, or without smoothing the borders).

A color bar 362 may be displayed in display 300, and may indicate average color of images or consolidated images in the stream. Time intervals may be indicated on a separate timeline, or on color bar 362, and may indicate the capture time of the images currently being displayed in window 340. A set of controls 314 may alter the display of the image stream in consolidated image window 340. Controls 314 may include for example stop, play, pause, capture image, step, fast-forward, rewind, or other controls, to freeze, speed up, or reverse the image stream in window 340. Viewing speed bar 312 may be adjusted by the user, for example the slider may indicate the number of displayed frames (e.g. consolidated frames or single frames) per second. Time indicator 310 may provide a representation of the absolute time elapsed for or associated with the current image being shown, the total length of the edited image stream and/or the original unedited image stream. Absolute time elapsed for the current image being shown may be, for example, the amount of time that elapsed between the moment the imaging device (e.g., capsule 40 of FIG. 1) was first activated or an image receiver (e.g., image receiver 12 of FIG. 1) started receiving transmission from the imaging device and the moment that the current image being displayed was captured or received.

Using control 316, a user may capture and store one or more of the currently displayed images as a thumbnail image (e.g. from the plurality of images which appear as a consolidated image in window 340) using an input device (e.g., mouse, touchpad, or other input device 24 of FIG. 1).

Thumbnail images 354, 356 may be displayed with reference to the appropriate relative frame capture time on the color bar (or time bar) 362. Related annotations or summaries 355, 357 may include the image capture time for each thumbnail image, and summary information associated with the current thumbnail image.

Capsule localization window 350 may include a current position and/or orientation of the imaging device in the gastrointestinal tract of the patient, and may display different segments of the GI tract is different colors. A highlighted segment may indicate the position of the imaging device during capture of the currently displayed image (or plurality of images). A progress bar or chart 352 may indicate the total path length travelled by the imaging device, and may provide an estimation or calculation of the percentage of the path travelled at the time the presently displayed image was captured.

Control 322 may allow the viewer to select between a manual viewing mode, for example an unedited image stream, and an automatically edited viewing mode, in which the user may view only a subset of images from the stream edited according to predetermined criteria. View layout controls 323 allow the viewer to select between viewing the image stream in a single window (one image being displayed in window 340), or viewing a consolidated image comprising two images (dual), four images (quadruple), or a larger number of images (e.g. 9, 16) in mosaic view layout. The display preview control 321 may display to the viewer selected images from the original stream, e.g. images selected as interesting or with clinical value (QV), the rest of the images (CQV), or only images with suspected bleeding indications (SBI).

Image adjustment controls 324 may allow a user to change the displayed image properties (e.g. intensity, color, etc.), while zoom control 325 enables increasing or decreasing the size of the displayed image in window 340. A user may select which display portions to show (e.g. thumbnails, localization, progress bar, etc.) using controls 326.

Reference is now made to FIGS. 3A-3C, which depict exemplary consolidated dual image display windows 280, 281, 282 according to embodiments of the invention. In FIG. 3A, consolidated image 280 includes two image portions (or regions) 210 and 211, which correspond, respectively, to two original sequential images 201, 202 from the originally captured image stream. The original images 201, 202 are round and separate, while in the consolidated image 280 the original images are reshaped to selected shape (or template) of the image portions 210, 211. It is important to note that image portions (or regions) 210, 211 do not include portions (or regions) 230, 231, 250 and 251.

In one embodiment, in order to reshape the original (e.g., round) image to the selected template contour, distortion minimization mapping techniques, e.g. conformal mapping techniques or “mean-value coordinates” technique (e.g. “Mean Value Coordinates” by Michael S. Floater, http://cs.brown.edu/courses/cs224/papers/mean_value.pdf), may be applied. A conformal map transforms any pair of curves intersecting at a point in the region so that the mapped image curves intersect at the same angle. Known solutions exist for conformal mapping of images, for example, Tobin A. Driscoll's version 2.3 of Schwarz-Christoffel Toolbox (SC Toolbox) is a collection of M-files for the interactive computation and visualization of Schwarz-Christoffel conformal maps in MATLAB version 6.0 or later (the toolbox is available in http://www.math.udel.edu/˜driscoll/software/SC/).

Other methods of distortion minimization mapping may be used. For example, the “As Rigid As Possible” (ASAP) technique is a morphing technique that blends the interiors of given two- or three-dimensional shapes rather than their boundaries. The morph is rigid in the sense that local volumes are least-distorting as they vary from their source to target configurations. One implementation of the “as rigid as possible” technique is disclosed in the article “As-Rigid-As-Possible Shape Interpolation” to Alexa, Cohen-Or and Levin, or “As-Rigid-As-Possible Shape Manipulation” to T. Igarashi, T. Moscovich and J. F. Hughes. Another technique, named “As Similar As Possible”, is described for example in Levi, Z. and Gotsman, C.'s “D-Snake: Image Registration by As-Similar-As-Possible Template Deformation”, published in IEEE Transactions on Visualization and Computer Graphics, 2012. Other techniques are possible, e.g. holomorphic mapping and quasi-conformal mapping.

A distortion minimization mapping may be computationally intensive, and thus in some embodiments the distortion minimization mapping calculation may be performed once, off-line, before in vivo images are displayed to a viewer. The computed map may be later applied to image streams gathered from patients, and the mapping may be applied during the image processing. A distortion minimization mapping transformation may be computed, for example, from a canonical circle to the selected template contour, e.g. rectangle, hexagon or any other shape. This initial computation may be done once, and the results may be applied to images captured by each capsule used. The computation may be applied to every captured frame. Online computation may also be used in some embodiments.

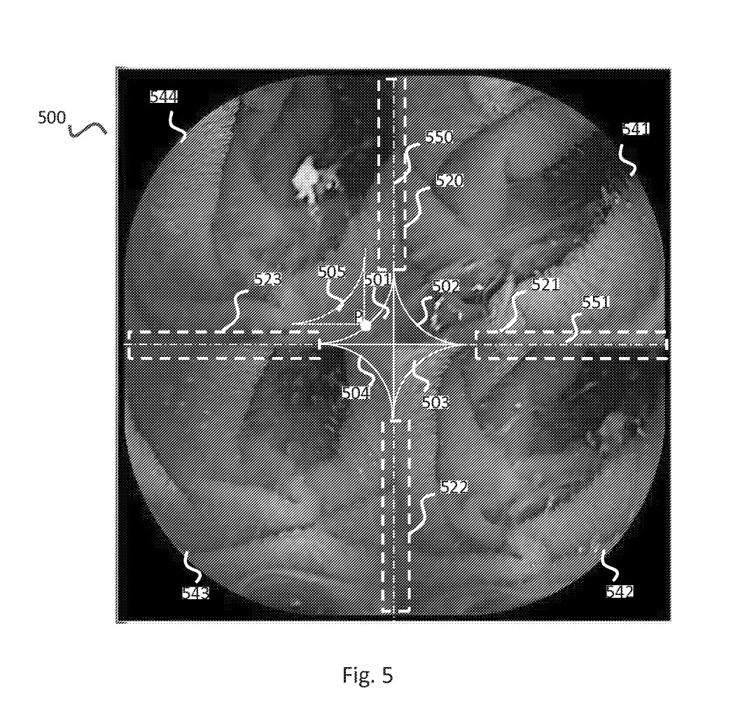

A need for filling regions or portions of an image may arise because if the original image shape is transformed into a different shape (e.g., a round image may be transformed to a shape with corners in case of a quadruple consolidated image as shown in FIG. 5), conformal mapping will generate large magnification of the original image at the corners of the transformed image. Thus, rounded corners may be used (instead of straight corners) in the image portion template, and empty portions or portions of the consolidated image, created as a result of the rounded corners, may be filled or generated.

A distortion minimization mapping algorithm may be used to transfer an original image to a differently-shaped image, e.g. original image 201 may be transformed to corresponding mapped image portion 210, and original image 211 to corresponding mapped image portion 202. In some embodiments, after the original image 201 is mapped to image portion 210, remaining predetermined empty regions or portions 230 and 250 of the consolidated image template may be automatically filled or generated. Similarly, original image 202 may be mapped to image portion 211, and remaining predetermined empty portions 231 and 251 of the template may be automatically filled or generated.

Fill may be, for example, content to use to fill or copy a portion of an image or a monitor display. Generating the fill for portions or regions 230, 250, or filling the regions, may be performed for example by copying a nearby patch or portion from mapped image portion 210 into the portions or regions to be generated or filled, and smoothing the edge created. Advantages of this method are that the local texture of a nearby patch is similar, and the motion direction is continuous. In an image stream created from consolidated images, since the patch is always copied from the same location in the original image, the flow of the video is continuous in the area of the generated portion or region, since the transitions between frames are locally identical to the transitions in a location the portion is copied from. This allows synthesizing sequential frames in the video independently, without checking the previous and/or subsequent frames, since the sequence of frames remains consistent and fluent.

In one embodiment, the patch may be selected, for example, such that the size and shape of the patch are identical to the size and shape of the portion or region which needs to be filled or generated. In other embodiments, the patch may be selected such that the size and/or shape of the patch are different from the size and shape of the region or portion which needs to be generated or filled, and the patch may be scaled, resized and/or reshaped accordingly to fit the generated portion or region.

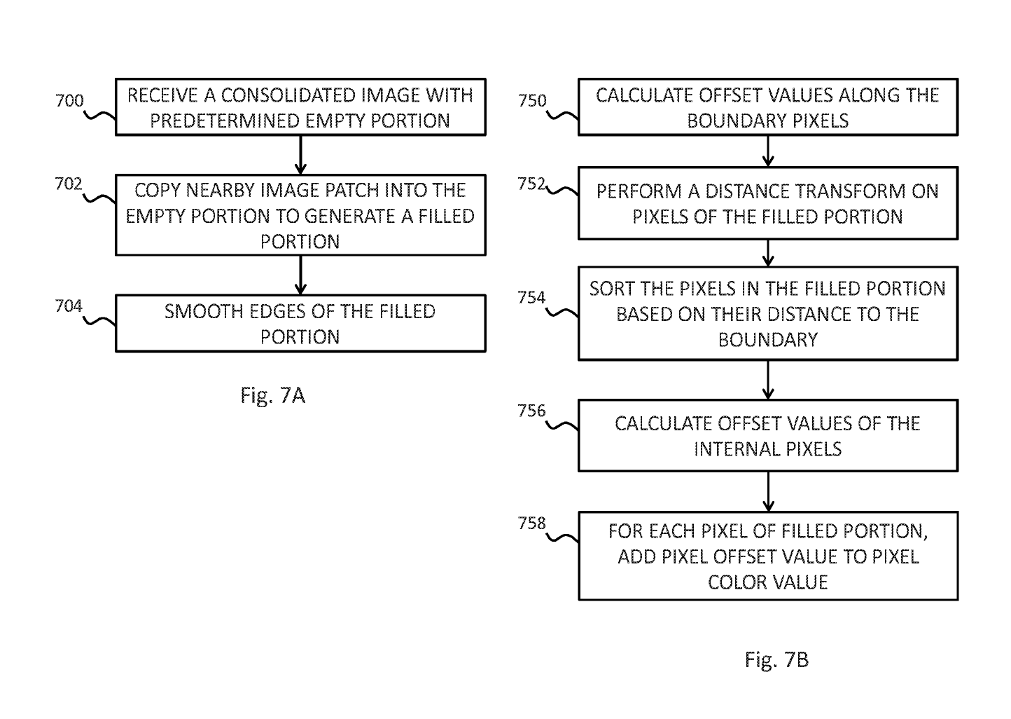

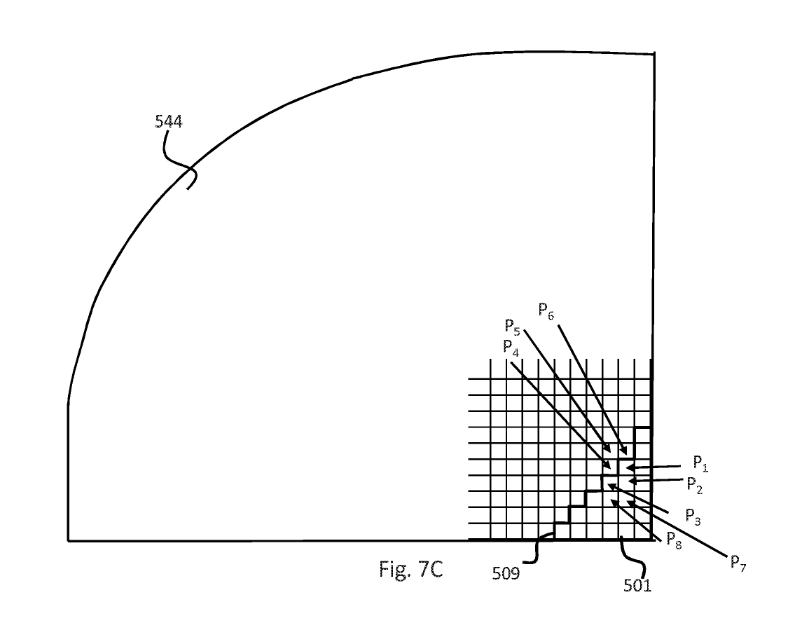

Synthesizing (or generating) regions or portions in consolidated images (which are displayed as part of an image stream) may require fast processing, e.g. in order to maintain the frame display rate of image stream, and to conserve processing resources for additional tasks. A method for smoothing edges of a filled (or generated) portion in a consolidated image is described in FIG. 7B herein.

Once the portions 230, 250 and 231, 251 are filled or generated, borders between the (mapped) image portions 210, 211 may be generated. The borders may be further processed using several methods. In one embodiment, the borders may be blended, smoothed or fused, and the two image portions 210, 211 may be merged into a single consolidated image with indistinct borders, e.g. as shown in region 220. In another embodiment, the borders may remain distinct, e.g. as shown in FIG. 3B, and a separation line 218 may be added to the consolidated image to emphasize the separation between the two image portions 212, 213. In yet another embodiment, a separation line need not be added, and the two image portions may simply be positioned adjacent each other, e.g. as indicated by edge 222 which shows the border between image portion 214 and image portion 215 in FIG. 3C. Edge 222 may define or be the border of the region or image portion 214, and the border may be made of pixels.

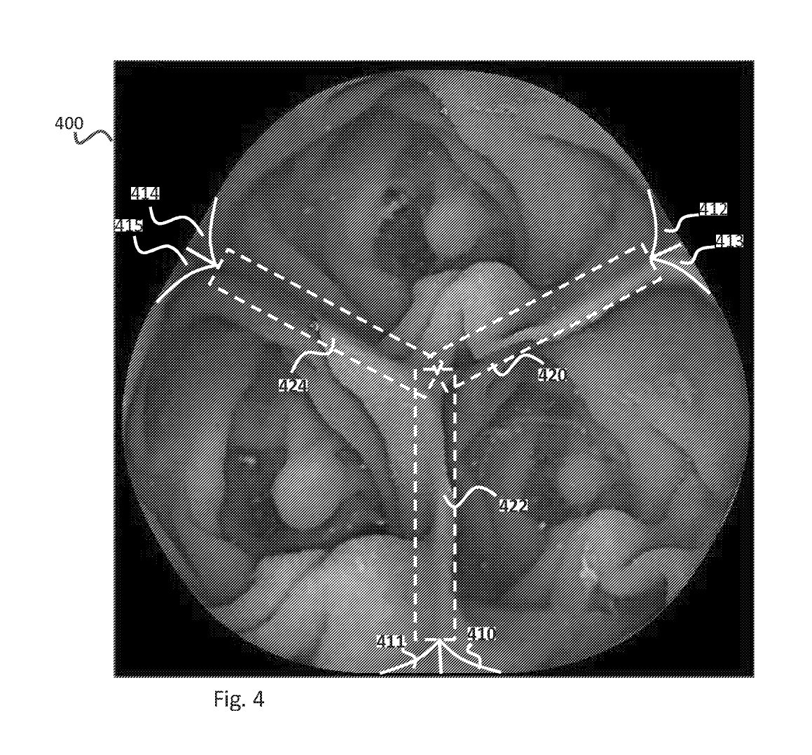

Reference is now made to FIG. 3D, which depicts an exemplary dual consolidated image template according to an embodiment of the present invention. Template 260 includes mapped image portions 270, 271, which are intended for mapping two original images selected for dual consolidated image display. Portions 261 and 262 are predetermined empty portions, which are intended to be generated or filled using a filling method as described herein. Portions 261 and 262 correspond to image portion 270, while portions 262 and 263 correspond to image portion 271. Line 273 indicates the separation between image portion 270 and image portion 271. Reference is now made to FIG. 4, which depicts an exemplary consolidated triple image display according to embodiments of the invention. Consolidated image 400 includes three image portions 441, 442 and 443, which correspond, respectively, to three original images from the captured image stream. The original images may be, for example, round and separate (e.g. similar to images 201 and 202 in FIG. 3A), while in the consolidated image 400 the original images are reshaped to the selected shape (or template) of the image portions 441, 442 and 443. Original images may also be shaped in any other shape, e.g. square, rectangular, etc.

Similar to the description of FIG. 3A above, in order to map or reshape the original (e.g., round) images 401, 402, 403 to the selected template shape of image portions 441, 442 and 443, distortion minimization techniques may be applied. Portions 410-415 may remain empty after mapping the original images to the new shape or contour of image portions 441, 442 and 443. Portions 410-415 may be generated or filled, for example as described with relation to portions 230, 231, 250 and 251 of FIG. 3A.

Once the portions 410-415 are filled or generated, borders between the image portions 441, 442 and 443 may be generated, using several methods. In one embodiment, the borders may be smoothed or fused, and the three image portions 441, 442 and 443 may be merged into a single consolidated image with indistinct borders, e.g. as shown in regions 420, 422 and 424. In another embodiment, the borders may remain distinct, e.g. as shown in FIG. 3B, with a separation line to emphasize the separation between the three image portions 441, 442 and 443. In yet another embodiment, a separation line need not be added, and the three image portions may simply be positioned adjacent each other, e.g. similar to edge 222 which indicates or is the border between image portion 214 and image portion 215 in FIG. 3C.

Reference is now made to FIG. 5, which depicts an exemplary consolidated quadruple image display according to embodiments of the invention. The rounded contour of consolidated image 500 may improve the process of viewing the image stream, e.g. due to better utilization of the human field of view. The resulting consolidated image may be more convenient to view, e.g. compared to original image contour such as round or rectangular. Consolidated image 500 includes four image portions 541, 542, 543, and 544 which correspond, respectively, to four original images from the captured image stream. Image portions 541-544 are indicated by axis 550 and axis 551, which divide the consolidated image 500 to four sub-portions, corresponding to the original image which was used to generate each portion. The original images are shaped different from the predetermined shape of the image portions 541, 542, 543, and 544. The position of images on consolidated image 500 may be defined by a template which determines where the mapped images appear, when they are applied to the template.

In this example, the original images are mapped to image portions 541-544, e.g. using conformal mapping techniques. It is important to note that image portions 541-544 do not include the internal portions or regions 501-504, which are intended to remain empty after the conformal mapping process. The reason is that if the same conformal mapping technique is used to map the original images to these portions as well, the mapping process may generate large magnifications at the corner areas (indicated by internal portions 501-504), and may create a distorted view of the proportions between objects captured in original images.

Internal portions 501-504 may be generated or filled by a filling technique, e.g. as described with relation to FIG. 3A. Borders between adjacent mapped image portions (e.g. between mapped image portions 541 and 542, or 541 and 544) may be smoothed (e.g. as shown in FIG. 5), separated by a line, or may remain as touching images with no distinct separation.

Once inner portions 501-504 are generated or filled, borders between the mapped image portions 541-544 may be generated, using one or more of several methods. In one embodiment, the borders may be smoothed or fused, and the four mapped image portions 541-544 may be merged into a single consolidated image with indistinct borders, e.g. as shown in connecting regions 520-523. In another embodiment, the borders may remain distinct, e.g. as shown in FIG. 3B, with a separation line to emphasize the separation between mapped image portions 541-544. In yet another embodiment, a separation line need not be added, and the four image portions may simply be positioned adjacent each other, e.g. similar to edge 222 which indicates the border between mapped image portion 214 and mapped image portion 215 in FIG. 3C. Other methods may be used.

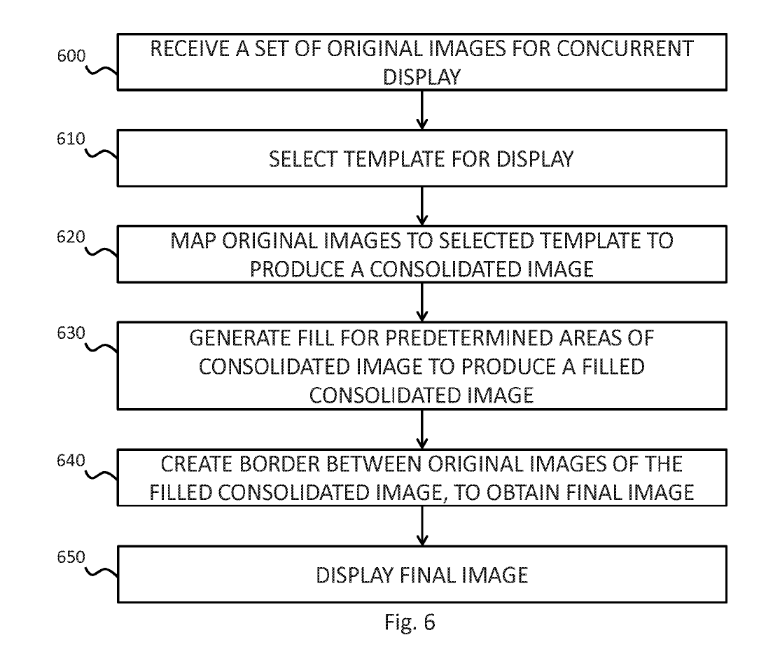

Reference is now made to FIG. 6, which is a flowchart depicting a method for displaying a consolidated image according to an embodiment of the invention. In operation 600, a plurality of original images may be received (e.g., from memory, or from an in-vivo imaging capsule) for concurrent display, e.g., display at the same time or substantially simultaneously, on the same screen or presentation. The plurality of original images may be selected for concurrent display as a consolidated image, the selection being from an image stream which was captured in vivo, e.g. by a swallowable imaging capsule. In one embodiment, the plurality of images may be chronologically-ordered sequential images, captured by the imaging capsule as it traverses the GI tract. The original images may be received, for example from a storage unit (e.g. storage 19) or image database (e.g. image database 21). The number of images in the plurality of images for concurrent display may be predetermined or automatically determined (e.g. by processor 14 or display generator 24), or may be received as input from the user (who may select, for example, dual, triple, or quadruple consolidated image display).

After the number of images to be concurrently displayed in a consolidated image is determined, a template for display may be selected or created in operation 610, e.g. automatically by a processor (such as processor 14 or display generator 24), or based on input from the user. The selected template may be selected from a set of predefined templates, stored in a storage unit (e.g. storage 19) which is operationally connected to the processor. In one embodiment, several predefined configurations may be available, e.g. one or more templates may be predefined per each number of images to be concurrently displayed on the screen as a consolidated image. In other embodiments, templates may be designed on the fly, e.g. according to user input such as the desired number of original images to consolidate and desired contour of the consolidated image.

The plurality of original images may be mapped or applied to the selected template, or mapped or applied to areas in the template, in operation 620, to produce a consolidated image. The consolidated image produced combines the plurality of original images into a single image with a predetermined contour. Each original image may be mapped or applied to one portion or area of the selected template. For example, the images may be mapped to the consolidated image portion according to an image property, e.g. chronological time of capture. In one example, the image from the plurality of original images which has the earliest capture time or capture timestamp may be mapped or applied to the left side of the template in dual view (e.g. to mapped image portion 210 in dual consolidated image of FIG. 3A). Other mapping arrangements may be selected, for example based on the likelihood of pathology captured in the image (e.g. the image with a highest pathology score or the image from the plurality of images for concurrent display which is most likely to include pathology).

In some embodiments, mapping the original images to predetermined portions of the selected template may be performed by conformal mapping techniques. Since conformal mapping preserves local angles of curves in the original images, the resulting transformed images maintain the shapes of objects (e.g. in vivo tissue) captured in the original images.

Conformal maps preserve angles and shapes of objects in the original image, but not necessarily their size. Mapping the original images may be performed according to various distortion minimization mapping techniques, such as “As Rigid As Possible” morphing technique, “As Similar As Possible” deformation technique, or other morphing or deformation methods as known in the art.

In some embodiments, the selected template may include predetermined areas of the consolidated images which remain empty after mapping the original images. These areas are not mapped due to intrinsic performance of the mapping algorithm, which may cause magnified corners in certain areas of the consolidated image. Therefore, a filling algorithm may be used to fill these areas in a manner which is useful to the reviewing professional (operation 630). The filled areas may be generated such that natural flow of the image stream is maintained when presented to the user. Different methods may be used to fill the predetermined empty areas of the consolidated image; one such method is presented in FIGS. 7A and 7B.

After the predetermined areas are filled using a filling algorithm, display generator 24 may generate the borders between the image portions (operation 640). Borders may be selected from different border types. The selected type of borders may be predetermined, e.g. set in a processor (e,g, processor 14, or display generator 24) or storage unit (e.g. storage 19), or may be manually selected by a user, via a user interface, according to personal preference. One type of borders may include separation lines, which may be added to the consolidated image to emphasize each image portion and to define the area to which each original image was mapped. Another option may include keeping the consolidated image without any explicit borders, e.g. no additional separation lines.

In another embodiment, the borders between image portions of the consolidated image may be blended, fused or smoothed, to create an indistinct transition from one image portion to another. For example, the smoothing operation may include image blending or cross-dissolve image merging techniques. One exemplary method is described in “Poisson Image Editing” by P′erez et al, which discloses a seamless image blending algorithm which determines the final image using a discrete Poisson equation.

After the borders are determined, the final consolidated image may be displayed to a user (operation 650), typically as part of an image stream of an in vivo gastrointestinal imaging procedure. The image may be displayed, for example on an external monitor or screen (e.g. monitor 18), which may be operationally connected to a workstation or computer which comprises, e.g., data processor 14, display generator 24 and storage 19.

Reference is now made to FIG. 7A, which is a flowchart depicting a method for generating or filling a predetermined empty portion or region in a consolidated image according to an embodiment of the invention. In operation 700, a processing unit (e.g. display generator 24) may receive a consolidated image with at least one predetermined empty portion, to which an original image was not mapped. For example, the consolidated image may be received after completing operation 620 of FIG. 6.

The contour or border of the predetermined empty portion may be acquired or determined, e.g. stored in the storage unit 19, and an image portion or patch having the same contour, shape or border may be copied from a nearby mapped image region of the consolidated image (operation 702). For example, in FIG. 5, predetermined empty portion 501 is filled using image patch 505, which is selected from the mapped image portion 544. It is noted that image patch 505 and portion 501 are of the same size and have the same contour, therefore copying image patch 505 into portion 501 does not require additional processing of the copied patch. The image patch may be selected from a fixed position in the corresponding mapped image portion, thus for each consolidated image, the position or coordinates of the image patch (which is copied into the empty portion) are known in advance. For example, the size and contour of the predetermined empty portion of the consolidated image template are typically predetermined (for example, this information may be stored along with the consolidated image template). Accordingly, the position, size and contour of the image patch to be selected from the mapped image portion may also be predetermined. After the patch 505 is copied into predetermined empty portion 501, the predetermined empty portion 501 becomes “generated portion” (or generated region or filled portion) 501.

In the example shown in FIG. 5, image patch 505 may be selected from the mapped image portion such that, for example, the bottom right corner P of the image patch 505 is adjacent (or touching) the boundary between image portion 544 and predetermined empty portion 501, and the rotation angle of image patch 505 in relation to predetermined empty portion 501 is zero. In other embodiments, different rotation angles of the image patch in relation to predetermined empty portion may be selected, and different coordinate positions of the image patch may be selected from the corresponding image portion. When the image patch is selected from the same region (e.g., same position, size and shape) in each consolidated image, the resulting generated portion is always obtained from the same coordinates in the mapped image portion, and the resulting video flow of the images in the consolidated image stream becomes smooth and fluent.

In some embodiments, the selected patch or region is not necessarily identical (e.g., in size and/or shape) to the predetermined empty portion. Typically, the selected patch may be similar in shape and size, however not necessarily identical. For example, a patch which is larger than the predetermined empty portion may be selected, and resized (and/or reshaped) to fit the predetermined empty portion. Similarly, the selected patch may be smaller than the predetermined empty portion, and may be resized (and/or reshaped) to fit the region. It is noted that if the selected patch is too large, the resizing may cause noticeable velocity differences in the video flow between sequential consolidated images, due to increased movement (between sequential images) of objects captured in the selected patch, compared to the movement or flow of objects captured in the mapped image portion.

In operation 704, the edges or borders created by placing or planting the copied patch or portion into the filled or generated portion in the consolidated image, may be smoothed, fused or blended, for example as described in FIG. 7B. Smoothing an edge created when a patch is copied to a generated, synthesized or filled portion may be performed in various methods. One approach, for example, is found in the article “Coordinates for Instant Image Cloning” by Zeev Farbman, Gil Hoffer, Yaron Lipman, Daniel Cohen-Or and Dani Lischinski, published in “Coordinates for instant image cloning”, ACM Transactions on Graphics 28(3) (Proc. ACM SIGGRAPH 2009), August 2009. The article introduces a coordinate-based approach, where the value of the interpolant at each interior pixel of the copied region is given by a weighted combination of values along the boundary. The approach is based on Mean-Value Coordinates (MVC). These coordinates may be expensive to compute, since the value of every pixel inside the boundary depends on all the boundary pixels.

Reference is now made to FIG. 7B, which is a flowchart depicting a method for smoothing edges of a filled, synthesized or generated portion in a consolidated image according to an embodiment of the invention. An offset value may be generated and assigned to each pixel in the synthesized or generated portion, in order to create a smooth edge between the mapped image portion to the generated or synthesized portion. The offset values of the pixels may be stored in the storage unit 19. For example, the following set of operations may be used (other operations may be used).

In the first phase, in operation 750, offset values of pixels of the generated portion which are adjacent the boundary pixels may be calculated. A boundary pixel may be a pixel among the pixels comprising the boundary between the synthesized or generated portion and the corresponding image portion. In one embodiment, boundary pixels may be pixels of the synthesized or generated portion which are adjacent pixels of the corresponding mapped image portion. In another embodiment, boundary pixels may be pixels of the mapped image portion, which are adjacent pixels of the corresponding synthesized or generated portion (but are not contained within the synthesized portion).

In the following embodiment, the boundary pixels are defined as pixels of the mapped image portion which are adjacent the generated or synthesized portion. The offset value of a pixel P_{A} in the generated portion, which is positioned adjacent a boundary pixel, may be calculated by finding the difference between a color value (which may comprise multiple color components such as red green and blue values, or a single component, i.e. intensity value) of at least one neighboring boundary pixel and the color value (e.g., R, G, B color values or intensity value) of the pixel P_{A}. A neighboring pixel may be selected from an area of the mapped image portion, near the generated portion 501 (e.g. an area contained in corresponding image portion 544 which is adjacent to the boundary 509, which indicated the boundary between mapped image portion 544 and generated portion 501).

The color value of a pixel may be represented in various formats as known in the art, e.g. using RGB, YUV or YCrCb color spaces. Other color spaces or color representations may be used. In some embodiments, not all color components are used for calculating the offset value of a pixel, for example only the red color component may be used if the pixels' color values are represented in RGB color space.

In one embodiment, more than one neighboring pixel may be selected for calculating the offset value of a pixel P_{A}, adjacent a boundary pixel. For example, the offset value of pixel P₁ which is adjacent boundary pixels in FIG. 7C may be calculated as the mean value of a plurality of neighboring boundary pixels (which are in mapped portion 544), e.g. three neighboring boundary pixels P₄, P₅ and P₆:

O(P₁)=⅓(c(P₄)+c(P₅)+c(P₆)),

where O(P₁) indicates the offset value of pixel P₁, and c(Pᵢ) indicates the color value of pixel Pᵢ.

A distance transform operation may be performed on pixels of the filled or generated portion (operation 752). The distance transform may include labeling or assigning each pixel of the generated portion with the distance (measured, for example, in pixels) to the boundary of the synthesized or generated portion or to the nearest boundary pixel. The distance values of the pixels may be stored in the storage unit 19. For example, FIG. 7C is an enlarged view of filled, synthesized or generated portion 501 and its corresponding image portion 544 shown in FIG. 5 (numerals of corresponding elements in FIGS. 5A and 7C are repeated). Boundary pixels of filled or generated portion 501 are positioned along boundary line 509. P₄, P₅ and P₆ are exemplary boundary pixels of generated portion 501, while P₁, P₂, P₃ and P₈ are exemplary pixels adjacent to boundary pixels. A neighboring pixel to a first pixel, as used herein, may include a pixel which is adjacent to, diagonal from, or touching the first pixel. The distance between, for example, pixel P₁ (which is a pixel in generated portion 501 adjacent boundary pixels P₄ and P₆), to the nearest neighboring boundary pixel P₄ (or P₆, both of which contained in mapped image portion 544), is one pixel. Therefore, in the distance transform operation, pixel P₁ is assigned the distance value 1. Similarly, the distance values of pixels P₂, P₃ and P₈ are assigned the distance value 1. The distance values are stored per pixel of the filled or generated portion, for example in storage unit 19.

In operation 754, the pixels in the filled, synthesized or generated portion 501 may be sorted according to their calculated distance from the boundary of the filled or generated portion (using the result of the distance transform operation). The sorting may be performed only once, and used for every consolidated image, such that each pixel positioned in a certain coordinate in the template, receives a fixed or permanent sorting value, e.g. corresponding to its calculated distance from the boundary. The next operations may be performed on each pixel, according to the sorting value of the pixel. For example, calculating the offset values of internal pixels as explained in operation 756, may be performed according to the sorted order. The sorting values of each pixel in the generated portion may be stored, e.g. in storage 19. The sorting may be from the smallest distance to the largest distance of the pixel from the boundary line 509.

In a second phase of offset value calculation, in operation 756, the pixels inside generated portion 501 (which may be referred to as “internal pixels” of the generated portion, and include all pixels of the generated portion except the pixels immediately adjacent the boundary pixels, e.g. pixels which received the value “1” in the distance transform) may be scanned or analyzed, e.g. according to the sorted order computed in operation 754. The offset value of each internal pixel may be calculated based on, for example, the offset value of at least one neighboring pixel, which had already been assigned an offset value. The offset values of the internal pixels may be stored in the storage unit 19.

The order in which the offset values for internal pixels is calculated may be by starting the calculation from the internal pixels nearest the boundary pixels (pixels whose distance from the boundary is minimal, e.g. the distance is less than two pixels) and gradually increasing distance from the boundary pixels. Offset values of the internal pixels may be computed as based on one or more neighboring pixels, which had already been assigned an offset value. The calculation may include computing a mean, average, weighted average or generalized mean of the offset values of the selected neighboring pixel(s) which had already been assigned an offset value, multiplied by a decay factor (e.g. 0.9 or 0.95). For example, the offset value of internal pixel P₇, which has a distance of two pixels from the boundary 509, may be computed by:

O(P₇)=½(O(P₈)+O(P₂))D, (eq. 2)

where O(Pᵢ) indicates the offset value of pixel Pᵢ, and D is the decay factor. Since P₈ and P₂ are pixels adjacent to boundary pixels, their offset value may be calculated in the first phase, e.g. as described in operation 750. Therefore, these pixels already have an offset value assigned to them, and the offset value of the internal pixels with a distance of two pixels from the boundary line 509 may be computed. Other pixels may be used for calculating the offset value, for example, only a single neighboring pixel may be used (e.g. only P₈, only P₂ or only P₃), or three or more neighboring pixels may be used.

The purpose of the decay factor is to have the offset values of internal pixels in the generated portion, which are positioned relatively far from the boundary, converge to 0, in order to cause a gradual transition of the colors in the generated portion to the original colors of the copied patch. The transition of colors from the pixels of the generated portion which are adjacent the boundary pixels, towards the pixels whose distance is furthest from the boundary, may become gradual, and this may create the smoothing or blending effect. Thus, the smoothing operation may be performed according to the sorted order, e.g. from the pixels adjacent the boundary pixels, towards the internal pixels which are farthest from the boundary.

In operation 758, color values (e.g. RGB color values or intensity values) of each pixel in the generated portion may be added to the corresponding offset value of the pixel to generate a new pixel color value, and the new pixel color value may be assigned to the pixel. The new pixel color values may be stored per pixel, for example in storage 19. The color values of the pixels in the generated portion may thus be gradually blended with colors of the image portion which is adjacent to the boundary, to obtain smoothed or blended edges between the image portion and the generated portion.

Since the generated (or filled, or synthesized) portion may be a fixed, predetermined area in the consolidated image template, operations 752 and 754 above may be performed only once and used for all consolidated image frames of any image stream.

One advantage of an embodiment of the invention is computation speed. For each pixel, eight values at most (if all surrounding neighboring pixels are used) may be averaged, and in practice the number of neighboring pixels with assigned offset values may be significantly less (e.g. three or four neighboring pixels). Furthermore, the entire sequence of averaging can be determined offline.

Other blending or smoothing methods may be used in addition or instead of the described method, e.g. cross-dissolve, discrete Poisson equation, etc. Other sets of operations may be used. Features of certain embodiments may be used with other embodiments shown herein.

The system and method of the present invention may allow an image stream to be viewed in an efficient manner and over a shorter time period. It will be appreciated by persons skilled in the art that the present invention is not limited by what has been particularly shown and described hereinabove. Rather the scope of the invention is defined by the claims that follow:

1. A method for synthesizing a portion in a consolidated image, the consolidated image comprising a mapped image portion and a generated portion, the mapped image portion comprising boundary pixels, and the generated portion comprising pixels adjacent to the boundary pixels and internal pixels, the method comprising:
performing a distance transform for the pixels of the generated portion to calculate, for each pixel, the distance of the pixel to the nearest boundary pixel;
calculating offset values of pixels in the generated portion which are adjacent to the boundary pixels;
calculating offset values of internal pixels in the generated portion based on the offset values of at least one neighboring pixel which had been assigned an offset value;
for each pixel in the generated portion, adding the calculated offset value of the pixel to the color value of the pixel to obtain a new pixel color value.

2. The method of claim 1 comprising:
receiving a set of original images from an in vivo imaging capsule for concurrent display; and
selecting a template for displaying the set of images, the template comprising at least a mapped image portion and a generated portion.

3. The method of claim 2 comprising:
mapping the original images to the mapped image portion in the selected template.

4. The method of claim 3 comprising:
generating fill for predetermined areas of the consolidated image to produce the generated portion of the consolidated image.

5. The method of claim 4 wherein the generating is performed by copying a patch from the mapped image portion to the generated portion.

6. The method of claim 1 comprising displaying the consolidated image, the consolidated image comprising the mapped image portion and the generated portion with the new pixel color values.

7. The method of claim 1 comprising sorting pixels in the generated portion based on the calculated distance; and calculating the offset values of internal pixels according to the sorted order.

8. The method of claim 1 wherein the boundary pixels of the mapped image portion comprise pixels which are adjacent pixels of the corresponding generated portion.

9. The method of claim 1 wherein calculating offset values of a pixel P_{A} in the generated portion, adjacent to a boundary pixel, is by computing the difference between a color value of P_{A} and a mean, median, generalized mean or weighted average of at least one neighboring pixel, the neighboring pixels selected from the boundary pixels adjacent to P_{A}.

10. The method of claim 1 wherein calculating offset values of an internal pixel in the generated portion is by computing the mean, median, generalized mean or weighted average of at least one neighboring pixel which has been assigned an offset value, times a decay factor.

11. A system for displaying a consolidated image, the consolidated image comprising a mapped image portion and a generated portion, the mapped image portion comprising boundary pixels, the generated portion comprising pixels adjacent to the boundary pixels and internal pixels, the system comprising:
a processor to:
calculate, for each pixel of the generated portion, a distance value of the pixel to the nearest boundary pixel;
calculate offset values of the pixels of the generated portion which are adjacent the boundary pixels;
calculate offset values of internal pixels in the generated portion based on the offset values of at least one neighboring pixel which had been assigned an offset value;
for each pixel in the generated portion, adding the calculated offset value of the pixel to the color value of the pixel to obtain a new pixel color value;
a storage unit to store the distance values, the offset values, and the new pixel color values;
and
a display to display the consolidated image, the consolidated image comprising the mapped image portion and the generated portion with the new pixel color values.

12. The system of claim 11 wherein the storage unit is to store a set of original images from an in vivo imaging capsule for concurrent display.

13. The system of claim 12 wherein the processor is to select a template for displaying the set of images, the template comprising at least a mapped image portion and a generated portion.

14. The system of claim 12 wherein the processor is to map the original images to the mapped image portion in the selected template to produce the mapped image portion.

15. The system of claim 12 wherein the processor is to generate fill for predetermined areas of the consolidated image to produce the generated portion.

16. The system of claim 15 wherein the processor is to generate fill by copying a patch from the mapped image portion to the generated portion.

17. The system of claim 11 wherein the processor is to sort pixels in the generated portion based on the calculated distance value, and to calculate the offset values of internal pixels according to the sorted order.

18. A method of deforming multiple images of a video stream to fit a human field of view, the method comprising:
using a distortion minimization technique to deform an image to a new contour based on a template pattern, the template pattern having rounded corners and an oval-like shape; and
displaying the deformed images as a video stream.

19. The method of claim 18 wherein the template pattern comprises a mapped image portion and a synthesized portion.

20. The method of claim 19 wherein the border between the mapped image portion and the synthesized portion is calculated by:
performing a distance transform for the pixels of the synthesized portion to calculate, for each pixel, the distance of the pixel to the nearest boundary pixel;
calculating offset values of pixels in the synthesized portion which are adjacent to boundary pixels, said boundary pixels located in the mapped image portion and adjacent pixels of the synthesized portion;
calculating offset values of internal pixels in the synthesized portion based on the offset values of at least one neighboring pixel which had been assigned an offset value;
for each pixel in the synthesized portion, adding the calculated offset value of the pixel to the color value of the pixel to obtain a new pixel color value.
